(19) United States
(12) Patent Application Publication
(10) Pub. No.: US 2008/0160099 A1
(43) Pub. Date: Jul. 3, 2008

(54) Methods For Treating or Preventing a Vascular Disease

(75) Inventors: Peter D. Collin, Sunset, ME (US); Martin D. Tilson III, Scardale, NY (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 11/667,326

(22) PCT Filed: Nov. 9, 2005
(86) PCT No.: PCT/US05/40875

§ 371 Date: Mar. 3, 2008

Related U.S. Application Data
(60) Provisional application No. 60/626,732, filed on Nov. 10, 2004.

(51) Int. Cl. A61K 35/56; A61K 31/353; A61K 31/715

(52) U.S. Cl. 424/574; 514/456; 514/54; 424/520

(57) ABSTRACT

The present invention relates to green tea and sea cucumber extracts, compositions thereof, and methods of treating or preventing a cardiovascular disease, a peripheral vascular disease, or an aneurysm in a subject, the methods comprising administering to the subject an effective amount of a green tea extract, a sea cucumber extract, or a compound derived from a green tea extract or a sea cucumber extract.

The invention disclosed herein was made with U.S. Government support from the National Heart, Lung, and Blood Institute/National Institutes of Health (HL64334). Accordingly, the U.S. Government has certain rights in this invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### 1. FIELD OF THE INVENTION

The present invention relates to green tea and sea cucumber extracts, compositions thereof, and methods of treating or preventing a cardiovascular disease, a peripheral vascular disease, or an aneurysm in a subject, the methods comprising administering to the subject an effective amount of a green tea extract, a sea cucumber extract, or a compound derived from a green tea extract or a sea cucumber extract.

### 2. BACKGROUND OF THE INVENTION

Vascular diseases are the leading cause of morbidity and mortality in the United States and most western countries. According to recent statistics, vascular disease accounted for about twice as many deaths in the U.S. compared to cancer, and about ten times as many deaths as accidents. Atherosclerosis is the most common vascular disease, but the term “vascular disease” encompasses a myriad of diseases, including stroke (both ischemic stroke, intracerebral hemorrhage, and subarachnoid hemorrhage), transient ischemic attack, systolic dysfunction, diastolic dysfunction, aneurysm (including aortic dissections), myocardial ischemia (also called “coronary artery disease”), angina pectoris, myocardial infarction, congestive heart failure, cardiomyopathy (including dilated congestive cardiomyopathy, hypertrophic cardiomyopathy, and restrictive cardiomyopathy), cor pulmonale, arrhythmias, valvular heart disease, endocarditis, and peripheral vascular disease (including peripheral arterial occlusion and venous thrombosis).

An aneurysm is a common vascular dysfunction resulting from the abnormal widening of a blood vessel. Typically, vascular aneurysms are formed as a result of the weakening of the wall of a blood vessel and subsequent ballooning of the vessel wall. Aneurysms may form in numerous location though the body, including the brain, the abdomen, and throughout the circulatory system. Aneurysms are generally treated using surgical techniques or alternatively, procedures involving the placement of a clamp or similar device across the neck of the aneurysm, thereby excluding the aneurysm from the blood flow.

An abdominal aortic aneurysm (“AAA”) involves a dilation, stretching, or ballooning of the abdominal aorta. The abdominal aorta is the portion of the aorta located within the abdominal cavity. It functions to carry blood from the heart to the lower extremities and abdominal organs. Typically, the abdominal aorta has a diameter of about 2 cm to about 2.5 cm in an adult and extends from the heart towards the groin, bifurcating into the iliac arteries to supply blood to the legs.

An AAA typically occurs between the renal arteries or immediately above the iliac arteries. The exact cause is unknown, but risk factors include atherosclerosis and hypertension. AAA may also be caused by infection, congenital weakening of the connective tissue component of the artery wall, or trauma.

Each year, approximately 200,000 people in the United States are diagnosed with AAA. Of those 200,000, nearly 15,000 may have AAA threatening enough to cause death if not treated. AAA can affect persons of any age or sex, but it is most often seen in men aged 40 to 70. If untreated, the wall of the aorta may progressively dilate and possibly rupture, resulting in severe internal bleeding, and possibly death. Other complications associated with AAA include formation of clots which can travel to other organs, and aortic dissection, which occurs when the lining of the artery tears and blood leaks into the wall of the artery.

Traditional treatment of AAA is surgical and involves replacing the affected portion of the aorta through a large abdominal incision. This procedure requires general anesthesia, an average hospital stay of five to 12 days, and a prolonged recovery period of from weeks to months. Newer, endovascular repair techniques generally involve placing a device, such as one or more stents and/or grafts across the aneurysm through the vasculature rather than via an open surgical procedure. Problems associated with the use of stents and grafts include leakage of blood around the stent-graft device, slippage of the device, and stress damage on the device caused by the subject's movement, which may result in breakage of the device. Thus, there is a need in the art for non-invasive methods to treat patients diagnosed with AAA. Such methods would provide major benefits for the patient, including treatment on an outpatient basis, less discomfort, minimized complications and a faster recovery time.

The matrix metalloproteinases (MMPs) are members of a family of at least 15 zinc-dependent endopeptidases that function extracellularly. The MMP family of enzymes contributes to both normal and pathological tissue remodeling. MMPs play a key role in the migration of normal and malignant cells through the body. They also act as regulatory molecules, both by functioning in enzyme cascades and by processing matrix proteins, cytokines, growth factors and adhesion molecules to generate fragments with enhanced or reduced biological effects.

In normal physiology, MMPs produced by connective tissue are thought to contribute to tissue remodeling in development, in the menstrual cycle, and as part of repair processes following tissue damage. The obvious destructive capability of MMPs initially focused most research onto diseases that involve breakdown of the connective tissues (e.g., rheumatoid arthritis, cancer and periodontal disease). Leukocytes, particularly macrophages, are major sources of MMP production. MMPs released by leukocytes play vital roles in allowing leukocytes to extravasate and penetrate tissues, a key event in inflammatory disease. It is proposed that MMP action not only permits leukocyte emigration into tissues and causes tissue damage, it also generates immunogenic fragments of normal proteins that may escalate autoimmune disease. In an analogous way, metastatic cancer cells also use MMPs to get in and out of tissues and to establish a blood supply. It has been demonstrated that small-molecule MMP inhibitors can demonstrate efficacy in models of these diseases, reinforcing their central role in pathology.

Recent studies suggest that abnormal MMP activity may be associated with the formation of various types of aortic aneurysms, including thoracic aortic aneurysms and AAAs. Matrix metalloproteinase-2 (MMP-2) and matrix metalloproteinase-9 (MMP-9) have been specifically implicated in the development of AAA.

MMP-2, also known as gelatinase A, is a 72 kDA type IV collagenase produced by fibroblasts and is a member of a family of proteolytic enzymes that use metal for their catalytic mechanism. The enzyme binds two zinc ions and four calcium ions per subunit and is responsible for cleavage of gelatin type I and collagen types IV, V, VII and X. MMP-2 is believed to be the principle metalloproteinase in small aneurysms and is present in high levels during early aneurysm development. Thus, MMP-2 is an attractive therapeutic target for the chemotherapeutic treatment of AAAs.

### 3. SUMMARY OF THE INVENTION

In one aspect, the invention provides sea cucumber extracts and green tea extracts (each being an “Extract of the Invention”).

In another aspect, the invention provides a catcechin, a sulfated polysaccaride or a pharmaceutically acceptable salt thereof (each being a “Compound of the Invention”).

In a further aspect, the invention provides compositions comprising a sea cucumber extract or a green tea extract and another therapeutically active compound (collectively referred to as an “Composition of the Invention”).

The Extracts, Compounds and Compositions of the Invention are useful for treating or preventing a cardiovascular disease, a peripheral vascular disease, or an aneurysm (each being a “Condition”).

In another aspect, the invention provides methods for treating or preventing an a Condition in a subject, the methods comprising administering to the subject an effective amount of an Extract, Compound or Composition of the Invention.

The invention also relates to pharmaceutical compositions comprising a physiologically acceptable carrier or vehicle and an effective amount of a Compound, Extract or Composition of the Invention. The pharmaceutical compositions are useful for treating or preventing a Condition in a subject.

The present invention may be understood more fully by reference to the following detailed description and illustrative examples, which are intended to exemplify non-limiting embodiments of the invention.

### 4. BRIEF DESCRIPTION OF THE DRAWINGS

FIGS. 1a-c shows the in vivo effect of a green tea extract containing (−)-epigallocatechin gallate in a murine model of abdominal aortic aneurysm. FIG. 1a shows a cross-section of the aorta of a healthy control animal. FIG. 1b shows the cross-section of the aorta of an animal having an abdominal aortic aneurysm 10 days after treatment with a green tea extract containing 55% (−)-epigallocatechin gallate. FIG. 1c shows a 10× magnified view of the aorta of an untreated animal having an abdominal aortic aneurysm.

FIG. 2 shows a graphical depiction of the effect of various Compounds of the Invention on MMP-2 activity at concentrations of 10, 25, 75 and 100 μg/mL. The three lines in the graph represent: fucosylated chondroitin sulfate (red line), (−)-epigallocatechin gallate (blue line), and a 1:1 mixture (by weight) of (−)-epigallocatechin gallate and fucosylated chondroitin sulfate (white line).

FIGS. 3a-c shows the effect of illustrative Compounds of the Invention on gelatinase activity using gel electrophoresis on 10% polyacrylamide gel containing gelatin. As indicated in the upper X-axis of each figure, illustrative Compounds of the Invention were tested at 10, 25, 75 and 100 μg/mL for inhibition of gelatinase activity. FIG. 3a shows the effect of (−)-epigallocatechin gallate, FIG. 3b illustrates the effect of fucosylated chondroitin sulfate, and FIG. 3c illustrates the effect of a 1:1 mixture (by weight) of (−)-epigallocatechin gallate and fucosylated chondroitin sulfate.

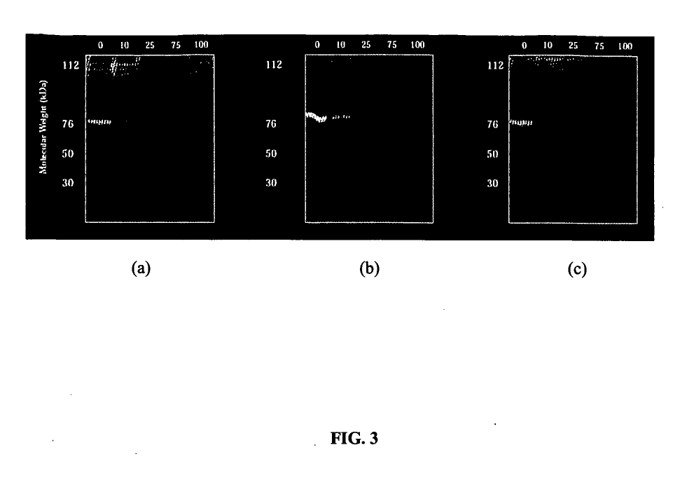

### 5. DETAILED DESCRIPTION OF THE INVENTION

The present invention relates to methods for treating and preventing a Condition in a subject, the methods comprising administering to a subject an effective amount of a Compound or Extract of the Invention.

### 5.1 Definitions

The terms used herein having following meaning:

The term “active component” as used herein, refers to any compound, fraction, or combination thereof, derived from a sea cucumber extract or a green tea extract as described herein, that is useful for treating or preventing a Condition. Illustrative examples of an active component of a green tea extract include, but are not limited to, a catechin, a bioflavanoid, a flavanol, a flavandiol, a tannin, and derivatives thereof. Illustrative examples of an active component of a sea cucumber extract includes, but is not limited to, a saponin, a sterol glycoside, a lactone, a lipid, a phospholipid, a peptide, a protamine, a glycogen, a polyphenol, a saccharide, a sulfated polysaccharide, and derivatives thereof.

The term “effective amount” when used in connection with a Compound, Extract, or Composition of the Invention is an amount that is effective to treat or prevent a Condition.

The term “in isolated form” as used herein means separated from other components of a reaction mixture or natural source. In certain embodiments, the isolate contains at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, or at least 98% of a Compound of the Invention or active component of an Extract of the Invention by weight of the isolate. In one embodiment, the isolate contains at least 95% of a Compound of the Invention by weight of the isolate. In one embodiment, the isolate contains at least 95% of an active component of an Extract of the Invention by weight of the isolate.

The phrase “pharmaceutically acceptable salt,” as used herein, is a salt formed from an acid and a basic nitrogen group of a Compound or Extract of the Invention. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-OH-3-naphthoate)) salts. The term “pharmaceutically acceptable salt” also refers to a salt prepared from a Compound or Extract of the Invention having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-hydroxy substituted lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxy lower alkyl)-amines, such as N,N-dimethyl-N-(2-hydroxyethyl)amine or tri-(2-hydroxyethyl)amine; N-methyl-D-glucamine; and amino acids such as arginine, lysine, and the like. The term “pharmaceutically acceptable salt” also includes a hydrate of a Compound or Extract of the Invention.

The term “sea cucumber” as used herein, refers to species of the Phylum Echinodermata, Class Holothuroidea. Illustrative sea cucumbers include species of the following genera: Actinopyga, Cucumaria, Eupentacta, Halodeima, Holothuria, Leptosynapta, Ludwigothurea, Microthele, Molpadia, Parastichopus, Paracaudina, Pelagothuria, Pentacta, Polycheira, Psolus, Stichopus, Synapta, Thelenota, and Thyone. In one embodiment, the sea cucumber belongs to the genus Cucumaria. In another embodiment, the sea cucumber belongs to the genus Ludwigothurea. In a specific embodiment, the sea cucumber is the species Ludwigothurea grisea. In another specific embodiment, the sea cucumber is the species Cucumariafrondosa.

The term “subject,” as used herein, includes, but is not limited to, a non-human animal, such as a cow, monkey, horse, sheep, pig, chicken, turkey, quail, cat, dog, mouse, rat, rabbit, or guinea pig; and a human. In one embodiment, a subject is a human.

The term “tensilin” refers to a protein which is isolated from the inner dermis of the sea cucumber.

The term “COX-2” refers to the enzyme cyclooxygenase-2.

The term “catechin” as used herein, is to be interpreted as being synonymous with the term “polyphenol.”

### 5.2 The Compounds, Extracts and Compositions of the Invention

The Compounds and Extracts of the Invention are useful for treating or preventing a Condition in a subject.

### 5.2.1 The Extracts of the Invention

Researchers, particularly those in the pharmaceutical industry, regularly screen extracts derived from natural sources in hopes of discovering biologically active compounds. Sea cucumber tissue and green tea leaves have been found to contain numerous biologically active compounds that are potentially useful in medical and veterinary applications.

Extracts from both the sea cucumber and green tea leaves are useful in the present methods for treating a Condition.

The Extracts of the Invention may be obtained from natural sources using extraction procedures well known to one of ordinary skill in the relevant art. The extraction procedures may be carried out using water, polar organic solvents, non-polar organic solvents, supercritical fluids, or mixtures thereof. Organic solvents useful for extracting an Extract of the Invention from a natural source include, but are not limited to alcohols, such as methanol, ethanol, n-propanol, isopropanol, n-butanol, isobutanol, sec-butanol and tert-butanol; ketones, such as acetone, methyl ethyl ketone, and ethyl acetate; ethers, such as diethyl ether, diphenyl ether, tetrahydrofuran, and dioxane; aliphatic hydrocarbons, such as pentanes, hexanes, and heptanes; aromatic hydrocarbons such as benzene, toluene, naphthalene, and xylenes; alkyl halides, such as carbon tetrachloride, choroform and methylene chloride; amides, such as dimethylformamide and hexamethylphosphoramide; carboxylic acids, such as formic acid and acetic acid; and sulfoxides, such as dimethylsulfoxide.

### 5.2.1.1 Green Tea Extracts

The present invention encompasses methods for treating a Condition in a subject, the methods comprising administering to the subject an effective amount of a green tea extract. The green tea extracts of the invention include both oil and water-soluble extracts and can be obtained from commercial sources (e.g., Nature's Resource, Mission Hills, Calif., or Herbasin, Beijing, China) or can be obtained directly from green tea leaves using extraction methods disclosed in European Patent No. EP 1402869 to Schneider, which is hereby incorporated by reference in its entirety.

Green Tea is known to contain numerous active components that are potentially useful in medical and veterinary applications, including catechins, bioflavanoids, flavanols, flavandiols, tannins, and derivatives thereof.

In one embodiment, a green tea extract contains from about 1% to about 90% catechins (by weight of the extract). In various embodiments a green tea extract contains about 10% catechins, about 20% catechins, about 30% catechins, about 40% catechins, about 50% catechins, about 60% catechins, about 70% catechins, about 80% catechins, and about 90% catechins.

In one embodiment, a green tea extract contains (−)-epigallocatechin gallate. In various embodiments, a green tea extract contains about 1% to about 90% (−)-epigallocatechin gallate (by weight of the extract). In various embodiments a green tea extract contains about 10% (−)-epigallocatechin gallate, about 20% (−)-epigallocatechin gallate, about 30% (−)-epigallocatechin gallate, about 40% (−)-epigallocatechin gallate, about 50% (−)-epigallocatechin gallate, about 60% (−)-epigallocatechin gallate, about 70% (−)-epigallocatechin gallate, about 80% (−)-epigallocatechin gallate, and about 90% (−)-epigallocatechin gallate.

In a specific embodiment, a green tea extract conatins about 55% (−)-epigallocatechin gallate.

Illustrative green tea extracts include the extracts described below.

Extract 8—a commercial green tea extract containing about 95% polyphenols (available from Herbasin, Shenyang, China).

### 5.2.1.2 Sea Cucumber Extracts

The present invention encompasses methods for treating a Condition in a subject, the methods comprising administering to the subject an effective amount of a sea cucumber extract. A sea cucumber extract may comprise one or more body parts from the sea cucumber, such as the skin, mouth, tentacles, body wall, muscle and viscera. Additionally, a sea cucumber extract may be obtained via an extraction of compounds contained within various parts of a sea cucumber, including but not limited to the sea cucumber's skin, mouth, tentacles, body wall, muscle and viscera. Methods useful in obtaining sea cucumber extracts from sea cucumber bodies are outlined in U.S. Pat. No. 5,985,330 to Collin.

Sea cucumbers are cylinder-shaped invertebrate animals that live in seas worldwide. There are approximately 900 species of sea cucumber in the taxonomic Class Holothuroidea. The dried or extracted sea cucumber is useful as a nutritional supplement and is known to contain numerous active components that are potentially useful in medical and veterinary applications. These active components include, but are not limited to, saponins, sterol glycosides, lipids, phospholipids, lactones, peptides, polyphenols, protamines, glycogens, saccharides, sulfated polysaccharides, and various amorphous compounds rich in saccharide moieties.

In one embodiment, a sea cucumber extract contains from about 1% to about 90% sulfated polysaccharides (by weight of the extract). In various embodiments a sea cucumber extract contains about 10% sulfated polysaccharides, about 20% sulfated polysaccharides, about 30% sulfated polysaccharides, about 40% sulfated polysaccharides, about 50% sulfated polysaccharides, about 60% sulfated polysaccharides, about 70% sulfated polysaccharides, about 80% sulfated polysaccharides, and about 90% sulfated polysaccharides.

In one embodiment, the sea cucumber extract is obtained from the inner dermis of the sea cucumber as described in U.S. Pat. No. 5,985,330 to Collin.

In one embodiment, the sea cucumber extract contains tensilin.

In another embodiment, the sea cucumber extract contains 12-methyltetradecanoic acid.

Illustrative sea cucumber extracts include the extracts described below.

Extract 1—a water extract of the epithelial layer of the sea cucumber Cucumaria frondosa, prepared as described below in Example 5.

Extract 2—a 70% isopropanol extract of the epithelial layer of the sea cucumber Cucumaria frondosa, prepared as described below in Example 6

Extract 3—an aqueous extract of the anterior portion of the body of the sea cucumber Cucumaria frondosa, prepared as described below in Example 7.

Extract 4—an aqueous extract of the anterior portion of the body of the sea cucumber Cucumaria frondosa, wherein the lipid components of the extract have been removed, prepared as described below in Example 8.

Extract 5—a supercritical carbon dioxide extract of intestinal tissue of the sea cucumber Cucumaria frondosa, prepared as described below in Example 9.

Extract 6—an ethanol extract of the body wall of the sea cucumber Cucumaria frondosa, prepared as described below in Example 5.

### 5.2.1.3 Compositions Comprising a Sea Cucumber Extract and/or a Green Tea Extract

The present invention also provides compositions comprising a green tea extract or a sea cucumber extract and one or more additional components. The compositions are useful for treating or preventing a Condition.

In one aspect, the present invention encompasses methods for treating a Condition in a subject, the methods comprising administering to the subject an effective amount of a Composition of the Invention.

In one embodiment, a Composition of the Invention comprises a green tea extract and a sea cucumber extract.

In another embodiment, a Composition of the Invention comprises comprises a green tea extract and one or more of the following compounds: a sea cucumber extract; a polyphenol, such as cis-resveratrol, trans-resveratrol, quercetin, a procyanidin or a prodelphidin; an omega-3 fatty acid, such as eicosapentaenoic acid or docosahexanoic acid; a matrix metalloproteinase inhibitor, such as doxycycline, marimistat or trocade; an angiogenesis inhibitor, such as angiostatin, endostatin, an interferon, interleukin 1 (and β), interleukin 12, retinoic acid, vitaxin, thalidomide, squalamine, or suramin; a lipoxygenase inhibitor, such as zileutin, and compounds disclosed in Rioux et al., Carcinogenesis, 19:1393-1400 (1998) and U.S. Pat. No. 6,465,421 to Duranton et al.; a COX-2 inhibitor, such as celecoxib, rofecoxib, valdecoxib, etoricoxib, parecoxib, piroxican, mefenamic acid, meloxican, nimesulfide, diclofenac, MF-tricyclide, raldecoxib, naproxen, or herbimycin-A; boswellia; glucosamine hydrochloride; soybean lecithin; fish oil, including one or more components thereof; or any compound or extract described in U.S. Pat. No. 6,541,519 to Collin.

In still another embodiment, a Composition of the Invention comprises comprises a sea cucumber extract and one or more of the following compounds: a polyphenol, such as cis-resveratrol, trans-resveratrol, quercetin, a procyanidin or a prodelphidin; an omega-3 fatty acid, such as eicosapentaenoic acid or docosahexanoic acid; a matrix metalloproteinase inhibitor, such as doxycycline, marimistat or trocade; an angiogenesis inhibitor, such as angiostatin, endostatin, an interferon, interleukin 1 (and β), interleukin 12, retinoic acid, vitaxin, thalidomide, squalamine, or suramin; a lipoxygenase inhibitor, such as zileutin, and compounds disclosed in Rioux et al., Carcinogenesis, 19:1393-1400 (1998) and U.S. Pat. No. 6,465,421 to Duranton et al.; a COX-2 inhibitor, such as celecoxib, rofecoxib, valdecoxib, etoricoxib, parecoxib, piroxican, mefenamic acid, meloxican, nimesulfide, diclofenac, MF-tricyclide, raldecoxib, naproxen, or herbimycin-A; boswellia; glucosamine hydrochloride; soybean lecithin; fish oil; or any compound or extract described in U.S. Pat. No. 6,541,519 to Collin.

In one embodiment, a Composition of the Invention comprises a green tea extract, a sea cucumber extract and eicosapentaenoic acid.

In another embodiment, a Composition of the Invention comprises a green tea extract, a sea cucumber extract and resveratrol.

In another embodiment, a Composition of the Invention comprises a green tea extract, a sea cucumber extract and tesilin.

In another embodiment, a Composition of the Invention comprises a green tea extract, a sea cucumber extract and 12-methyltetradecanoic acid.

In still another embodiment, a Composition of the Invention comprises a green tea extract, a sea cucumber extract and boswellia.

In a further embodiment, a Composition of the Invention comprises a green tea extract, a sea cucumber extract, eicosapentaenoic acid, resveratrol, boswellia, and fish oil triglycerides.

In another embodiment, a Composition of the Invention comprises a green tea extract, a sea cucumber extract and glucosamine hydrochloride.

In yet another embodiment, a Composition of the Invention comprises a green tea extract, a sea cucumber extract, eicosapentaenoic acid and soybean lecithin.

Illustrative examples of Compositions of the Invention are set forth below.

Composition 1—A composition consisting of: 40% eicosapentaenoic acid, 10% Extract 6, 10% Extract 8, 5% boswellia (obtained from the tree Boswellia serrata), and 5% resveratrol (obtained from Japanese knotweed (polygonum cuspidatum)), and 40% fish oil triglycerides, by total weight of the composition.

Composition 2—A composition consisting of: 40% Extract 8, 40% glucosamine hydrochloride, and 20% Extract 3, by total weight of the composition.

Composition 3—A composition consisting of: Extract 8 (10%), Extract 1 (20%), eicosapentaenoic acid (30%), and soybean lecithin (40%), by total weight of the composition.

### 5.2.2 Compounds of the Invention

The Compounds of the Invention include members of the classes of organic compounds commonly known as catechins and sulfated polysaccharides, derivatives thereof, and mixtures thereof. The Compounds of the Invention also include any active component that is present in a green tea extract or a sea cucumber extract. The Compounds of the Invention may be purchased from commercial sources (e.g., Sigma Chemical, St. Louis, Mo.), prepared synthetically using methods well-known to those of ordinary skill in the art of synthetic organic chemistry, or extracted from natural sources using methods well-known to those of skill in the arts of chemistry, biology or related arts.

In one embodiment, a Compound of the Invention is obtained from a sea cucumber extract. In another embodiment, a Compound of the Invention is obtained from a green tea extract.

It is possible for the Compounds of the Invention to have one or more chiral centers and as such the Compounds of the Invention can exist in various stereoisomeric. Accordingly, the present invention is understood to encompass all possible stereoisomers and geometric isomers.

The present invention also includes Compounds of the Invention wherein one or more hydrogen, carbon or other atoms are replaced by an isotope thereof. Such compounds are useful as research or diagnostic tools in metabolism pharmacokinetic studies, in binding assays, and as diagnostic imaging agents.

In one embodiment, a Compound of the invention is in isolated form. In one embodiment, the Compound of the invention is fucosylated chondroitin sulfate.

### 5.2.2.1. Catechins

As stated above, the present invention encompasses methods for treating a Condition in a subject, the methods comprising administering to the subject an effective amount of a catechin, a catechin derivative or a pharmaceutically acceptable salt of a catechin or catechin derivative. Illustrative catechins useful in the present methods for treating or preventing a Condition, include, but are not limited to the following compounds and pharmaceutically acceptable salts thereof: catechin, epicatechin, epicatechin gallate, gallocatechin gallate, epigallocatechin, and epigallocatechin gallate. These compounds are useful in the present methods alone, or in combination with one or more Compounds or Extracts of the Invention.

In one embodiment, the catechin is epigallocatechin gallate.

In a specific embodiment, the catechin is (−)-epigallocatechin gallate.

### 5.2.2.2 Sulfated Polysaccharides

As stated above, the present invention encompasses methods for treating a Condition in a subject, the methods comprising administering to the subject an effective amount of a sulfated polysaccharide, a derivative of a sulfated polysaccharide, or a pharmaceutically acceptable salt of a sulfated polysaccharide or sulfated polysaccharide derivative. Illustrative sulfated polysaccharides useful in the present methods for treating or preventing a Condition, include, but are not limited to the following compounds and pharmaceutically acceptable salts thereof: chondroitin sulfate, and fucosylated chondroitin sulfate. These compounds are useful in the present methods alone, or in combination with one or more Compounds or Extracts of the Invention.

In one embodiment, the sulfated polysaccharide is chondroitin sulfate.

In a specific embodiment, the sulfated polysaccharide is fucosylated chondroitin sulfate.

### 5.3 Uses of the Compounds and Extracts of the Invention

### 5.3.1 Treatment or Prevention of a Cardiovascular Disease

A cardiovascular disease can be treated or prevented by administration of an effective amount of a Compound or Extract of the Invention.

Cardiovascular diseases that can be treated or prevented by administering an effective amount of a Compound or Extract of the Invention include, but are not limited to, atherosclerosis; arteriosclerosis; stroke, including ischemic stroke, intracerebral hemorrhage, and subarachnoid hemorrhage; transient ischemic attack; systolic dysfunction; diastolic dysfunction; coronary artery disease; angina pectoris; myocardial infarction; congestive heart failure; cardiomyopathy, including dilated congestive cardiomyopathy, hypertrophic cardiomyopathy, and restrictive cardiomyopathy; cor pulmonale; an arrhythmia; valvular heart disease; endocarditis; pulmonary vascular disease; congenital heart disease; an inflammation of the aorta; and Takayasu's arteritis.

In one embodiment, the cardiovascular disease is atherosclerosis.

### 5.3.2 Treatment or Prevention of an Aneurysm

An aneurysm can be treated or prevented by administration of an effective amount of a Compound or Extract of the Invention.

Aneurysms that can be treated or prevented by administering an effective amount of a Compound or Extract of the Invention include, but are not limited to, aortic aneurysms, such as an abdominal aortic aneurysm, a thoracic aortic aneurysm; an intracranial aneurysm, such as, a congenital saccular aneurysm or a mycotic aneurysm; and a peripheral aneurysm, such as a poplitiac aneurysm, an iliac aneurysm, a femoral aneurysm, an upper extremity aneurysm, or a splanchnic artery aneurysm.

In one embodiment, the aneurysm is an aortic aneurysm.

In another embodiment, the aneurysm is a peripheral aneurysm.

In still another embodiment, the aneurysm is an intracranial aneurysm.

In a specific embodiment, the aneurysm is abdominal aortic aneurysm.

In one embodiment, the “treatment” of an aneurysm refers to the cessation of growth of the aneurysm.

In another embodiment, the “treatment” of an aneurysm refers to a reduction in the size of the aneurysm.

### 5.3.3 Treatment or Prevention of a Peripheral Vascular Disorder

A peripheral vascular disorder can be treated or prevented by administration of an effective amount of a Compound or Extract of the Invention.

Types of peripheral vascular disorders that can be treated or prevented by administering an effective amount of a Compound or Extract of the Invention include a peripheral artery disease, such as carotid artery disease, peripheral arterial disease of the lower extremities, peripheral arterial disease of the renal arteries, peripheral arterial occlusion, Reynaud syndrome, Buerger disease, and polyarteritis nodosa; and a peripheral venous disorder, such as thrombophlebitis, arteriovenous fistula, an occlusion of the abdominal aorta and it's branches, venous thrombosis, thromboangiitis obliterans, one or more varicose veins, and chronic venous insufficiency.

In one embodiment the peripheral vascular disorder is a peripheral artery disease.

In another embodiment, the peripheral vascular disorder is a peripheral venous disorder.

In another embodiment, the peripheral vascular disorder is aortic dissection.

### 5.4 Therapeutic/Prophylactic Administration and pharmaceutical Compositions of the Invention

Due to their activity, the Compounds and Extracts of the Invention are advantageously useful in veterinary and human medicine. As described above, the Compounds and Extracts of the Invention are useful for treating or preventing a Condition in a subject in need thereof.

When administered to a subject, the Compounds and Extracts of the Invention can be administered as a component of a composition that comprises a physiologically acceptable carrier or vehicle. The present compositions, which comprise a Compound or Extract of the Invention, can be administered orally. The Compounds and Extracts of the Invention can also be administered by any other convenient route, for example, by infusion or bolus injection, by absorption through epithelial or mucocutaneous linings (e.g., oral, rectal, and intestinal mucosa, etc.) and can be administered together with another biologically active agent. Administration can be systemic or local. Various delivery systems are known, e.g., encapsulation in liposomes, microparticles, microcapsules, capsules, etc., and can be administered.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result in the release of the Compounds and Extracts of the Invention into the bloodstream. The mode of administration is left to the discretion of the practitioner.

In one embodiment, the Compounds and Extracts of the Invention are administered orally.

In another embodiment, the Compounds and Extracts of the Invention are administered intravenously.

In other embodiments, it can be desirable to administer the Compounds and Extracts of the Invention locally. This can be achieved, for example, and not by way of limitation, by local infusion during surgery, topical application, e.g., in conjunction with a wound dressing after surgery, by injection, by means of a catheter, by means of a suppository or enema, or by means of an implant, said implant being of a porous, non-porous, or gelatinous material, including membranes, such as sialastic membranes, or fibers.

In certain embodiments, it can be desirable to introduce the Compounds and Extracts of the Invention into the central nervous system or gastrointestinal tract by any suitable route, including intraventricular, intrathecal, and epidural injection, and enema. Intraventricular injection can be facilitated by an intraventricular catheter, for example, attached to a reservoir, such as an Ommaya reservoir.

Pulmonary administration can also be employed, e.g., by use of an inhaler of nebulizer, and formulation with an aerosolizing agent, or via perfusion in a fluorocarbon or a synthetic pulmonary surfactant. In certain embodiments, the Compounds and Extracts of the Invention can be formulated as a suppository, with traditional binders and excipients such as triglycerides.

In another embodiment the Compounds and Extracts of the Invention can be delivered in a vesicle, in particular a liposome (see Langer, Science 249:1527-1533 (1990) and Treat or prevent et al., Liposomes in the Therapy of Infectious Disease and Cancer 317-327 and 353-365 (1989)).

In yet another embodiment the Compounds and Extracts of the Invention can be delivered in a controlled-release system or sustained-release system (see, e.g., Goodson, in Medical Applications of Controlled Release, supra, vol. 2, pp. 115-138 (1984)). Other controlled or sustained-release systems discussed in the review by Langer, Science 249:1527-1533 (1990) can be used. In one embodiment a pump can be used (Langer, Science 249:1527-1533 (1990); Sefton, CRC Crit. Ref Biomed. Eng. 14:201 (1987); Buchwald et al., Surgery 88:507 (1980); and Saudek et al., N. Engl. J Med. 321:574 (1989)). In another embodiment polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

In yet another embodiment a controlled- or sustained-release system can be placed in proximity of a target of the Compound or Extract of the Invention s, e.g., the spinal column, brain, heart, abdomen, thoracic cavity, skin, lung, or gastrointestinal tract, thus requiring only a fraction of the systemic dose.

The present compositions can optionally comprise a suitable amount of a physiologically acceptable excipient so as to provide the form for proper administration to the subject.

Such physiologically acceptable excipients can be liquids, such as water and oils, including those of petroleum, subject, vegetable, or synthetic origin, such as peanut oil, soybean oil, mineral oil, sesame oil and the like. The pharmaceutical excipients can be saline, gum acacia; gelatin, starch paste, talc, keratin, colloidal silica, urea and the like. In addition, auxiliary, stabilizing, thickening, lubricating, and coloring agents can be used. In one embodiment the physiologically acceptable excipients are sterile when administered to a subject. Water is a particularly useful excipient when the Compound or Extract of the Invention is administered intravenously. Saline solutions and aqueous dextrose and glycerol solutions can also be employed as liquid excipients, particularly for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills; pellets, capsules, capsules containing liquids, powders, sustained-release formulations, suppositories, emulsions, aerosols, sprays, suspensions, or any other form suitable for use. In one embodiment the composition is in the form of a capsule (see e.g. U.S. Pat. No. 5,698,155). Other examples of suitable physiologically acceptable excipients are described in Remington's Pharmaceutical Sciences 1447-1676 (Alfonso R. Gennaro eds., 19th ed. 1995), incorporated herein by reference.

In one embodiment the Compounds and Extracts of the Invention are formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving a Compound or Extract of the Invention are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment the excipients are of pharmaceutical grade.

In another embodiment the Compounds and Extracts of the Invention can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized-powder or water free concentrate in a hermetically sealed container such as an ampule or sachette indicating the quantity of active agent. Where the Compounds and Extracts of the Invention are to be administered by infusion, they can be dispensed, for example, with an infusion bottle containing sterile pharmaceutical grade water or saline. Where the Compounds and Extracts of the Invention are administered by injection, an ampule of sterile water for injection or saline can be provided so that the ingredients can be mixed prior to administration.

The Compounds and Extracts of the Invention can be administered by controlled-release or sustained-release means or by delivery devices that are well known to those of ordinary skill in the art. Examples include, but arc not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference. Such dosage forms can be used to provide controlled- or sustained-release of one or more active components using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active components of the invention. The invention thus encompasses single unit dosage forms suitable for oral administration such as, but not limited to, tablets, capsules, gelcaps, and caplets that are adapted for controlled- or sustained-release.

In one embodiment a controlled- or sustained-release composition comprises a minimal amount of a Compound or Extract of the Invention to treat or prevent a Condition in a minimal amount of time. Advantages of controlled- or sustained-release compositions include extended activity of the drug, reduced dosage frequency, and increased subject compliance. In addition, controlled- or sustained-release compositions can favorably affect the time of onset of action or other characteristics, such as blood levels of the Compound or Extract of the Invention, and can thus reduce the occurrence of adverse side effects.

Controlled- or sustained-release compositions can initially release an amount of a Compound or Extract of the Invention that promptly produces the desired therapeutic or prophylactic effect, and gradually and continually release other amounts of the Compound or Extract of the Invention to maintain this level of therapeutic or prophylactic effect over an extended period of time. To maintain a constant level of the Compound or Extract of the Invention in the body, the Compound or Extract of the Invention can be released from the dosage form at a rate that will replace the amount of Compound or Extract of the Invention being metabolized and excreted from the body. Controlled- or sustained-release of Compound of the Invention or an active component of an Extract of the Invention can be stimulated by various conditions, including but not limited to, changes in pH, changes in temperature, concentration or availability of enzymes, concentration or availability of water, or other physiological conditions or compounds.

The amount of the Compound or Extract of the Invention that is effective in the treatment or prevention of a Condition can be determined by standard clinical techniques. In addition, in vitro or in vivo assays can optionally be employed to help identify optimal dosage ranges. The precise dose to be employed will also depend on the identity of the Compound or Extract of the Invention, route of administration, and the seriousness of the condition being treated and should be decided according to the judgment of the practitioner and each subject's circumstances in view of, e.g., published clinical studies. Suitable effective dosage amounts for Compounds of the Invention, however, range from about 10 micrograms to about 5 grams. In certain embodiments, the effective dosage is about 0.01 mg, about 0.5 mg, about 1 mg, about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, and about 5.0 g. Dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months.

Suitable effective dosage amounts for the Extracts of the Invention are based upon the amount of active component(s) in the extract. For the Extracts of the Invention, the active component should be within a range of from about 0.01 to about 100 w/w %. In certain embodiments, the active component is within a range of from about 0.05 to about 80 w/w %, from about 1.0 to about 50 w/w %, from about 5 to about 30 w/w %, or from about 10 to about 20 w/w %. In certain embodiments, the effective dosage of the Extract of the Invention pertains to an amount of active component of about 0.01 mg, about 0.5 mg, about 1 mg, about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, and about 5.0 g. Dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months.

The effective dosage amounts described herein refer to total amounts administered; that is, if more than one Compound of the Invention is administered, the effective dosage amounts correspond to the total amount administered. If one or more Extract of the Invention is administered, the effective dosage amounts correspond to the total amount of the active components administered.

The Compounds and Extracts of the Invention can be assayed in vitro or in vivo for the desired therapeutic or prophylactic activity prior to use in humans. Subject model systems can be used to demonstrate safety and efficacy.

The present methods for treating or preventing a Condition in a subject can further comprise administering another therapeutic agent to the subject being administered a Compound or Extract of the Invention. In one embodiment the other therapeutic agent is administered in an effective amount. In another embodiment, the other therapeutic agent is another Compound or Extract of the Invention.

In another embodiment, the other therapeutic agent is an agent useful for the treatment of a cardiovascular disease.

In another embodiment the other therapeutic agent is an agent useful for the treatment of a peripheral vascular disease.

In a further embodiment, the other therapeutic agent is an agent useful for reducing any potential side effect of a Compound or Extract of the Invention.

Illustrative other therapeutic agents include, but are not limited to, a polyphenol, such as cis-resveratrol, trans-resveratrol, quercetin, a procyanidin or a prodelphidin; an omega-3 fatty acid, such as eicosapentaenoic acid or docosahexanoic acid; a matrix metalloproteinase inhibitor, such as doxycycline, marimistat or trocade; an angiogenesis inhibitor, such as angiostatin, endostatin, an interferon, interleukin 1 (and β), interleukin 12, retinoic acid, vitaxin, thalidomide, squalamine, or suramin; a lipoxygenase inhibitor, such as zileutin, and compounds disclosed in Rioux et al., Carcinogenesis, 19:1393-1400 (1998) and U.S. Pat. No. 6,465,421 to Duranton et al.; a COX-2 inhibitor, such as celecoxib, rofecoxib, valdecoxib, etoricoxib, parecoxib, piroxican, mefenamic acid, meloxican, nimesulfide, diclofenac, MF-tricyclide, raldecoxib, naproxen, or herbimycin-A; boswellia; glucosamine hydrochloride; soybean lecithin; fish oil; or any compound or extract described in U.S. Pat. No. 6,541,519 to Collin.

In one embodiment the other therapeutic agent is an angiogenesis inhibitor

In another embodiment the other therapeutic agent is a matrix metalloproteinase inhibitor.

In yet another embodiment the other therapeutic agent is an agent useful for the treatment of an aneurysm.

In a specific embodiment, the other therapeutic agent is tensilin.

In another specific embodiment, the other therapeutic agent is 12-methyltetradecanoic acid.

In another specific embodiment, the other therapeutic agent is resveratrol.

In another specific embodiment, the other therapeutic agent is eicosapentaenoic acid.

In still another specific embodiment, the other therapeutic agent is resveratrol.

In one embodiment, the present invention provides methods for treating or preventing a Condition in a subject comprising administering to the subject an effective amount of a green tea extract and an effective amount of a sea cucumber extract. In another embodiment, the present invention provides methods for treating or preventing a Condition in a subject comprising administering to the subject an effective amount of a green tea extract and an effective amount of an angiogenesis inhibitor.

In still another embodiment, the present invention provides methods for treating or preventing a Condition in a subject comprising administering to the subject an effective amount of a green tea extract and an effective amount of a lipoxygenase inhibitor.

In still another embodiment, the present invention provides methods for treating or preventing a Condition in a subject comprising administering to the subject an effective amount of a green tea extract and an effective amount of a COX-2 inhibitor.

In a further embodiment, the present invention provides methods for treating or preventing a Condition in a subject comprising administering to the subject an effective amount of a green tea extract and an effective amount of a matrix metalloproteinase inhibitor.

In yet another embodiment, the present invention provides methods for treating or preventing a Condition in a subject comprising administering to the subject an effective amount of a green tea extract and an effective amount of resveratrol.

In yet another embodiment, the present invention provides methods for treating or preventing a Condition in a subject comprising administering to the subject an effective amount of a green tea extract and an effective amount of 12-methyltetradecanoic acid.

In one embodiment, the present invention provides methods for treating or preventing a Condition in a subject comprising administering to the subject an effective amount of a sea cucumber extract and an effective amount of an angiogenesis inhibitor.

In still another embodiment, the present invention provides methods for treating or preventing a Condition in a subject comprising administering to the subject an effective amount of a sea cucumber extract and an effective amount of a lipoxygenase inhibitor.

In still another embodiment, the present invention provides methods for treating or preventing a Condition in a subject comprising administering to the subject an effective amount of a sea cucumber extract and an effective amount of a COX-2 inhibitor.

In a further embodiment, the present invention provides methods for treating or preventing a Condition in a subject comprising administering to the subject an effective amount of a sea cucumber extract and an effective amount of a matrix metalloproteinase inhibitor.

In yet another embodiment, the present invention provides methods for treating or preventing a Condition in a subject comprising administering to the subject an effective amount of a sea cucumber extract and an effective amount of resveratrol.

In one embodiment, the present invention provides methods for treating or preventing a Condition in a subject comprising administering to the subject an effective amount of (−)-epigallocatechin gallate and an effective amount of an angiogenesis inhibitor.

In still another embodiment, the present invention provides methods for treating or preventing a Condition in a subject comprising administering to the subject an effective amount of (−)-epigallocatechin gallate and an effective amount of a lipoxygenase inhibitor.

In still another embodiment, the present invention provides methods for treating or preventing a Condition in a subject comprising administering to the subject an effective amount of (−)-epigallocatechin gallate and an effective amount of a COX-2 inhibitor.

In a further embodiment, the present invention provides methods for treating or preventing a Condition in a subject comprising administering to the subject an effective amount of (−)-epigallocatechin gallate and an effective amount of a matrix metalloproteinase inhibitor.

In yet another embodiment, the present invention provides methods for treating or preventing a Condition in a subject comprising administering to the subject an effective amount of (−)-epigallocatechin gallate and an effective amount of resveratrol.

In yet another embodiment, the present invention provides methods for treating or preventing a Condition in a subject comprising administering to the subject an effective amount of (−)-epigallocatechin gallate and an effective amount of 12-methyltetradecanoic acid.

In one embodiment, the present invention provides methods for treating or preventing a Condition in a subject comprising administering to the subject an effective amount of fucosylated chondroitin sulfate and an effective amount of an angiogenesis inhibitor.

In still another embodiment, the present invention provides methods for treating or preventing a Condition in a subject comprising administering to the subject an effective amount of fucosylated chondroitin sulfate and an effective amount of a lipoxygenase inhibitor.

In still another embodiment, the present invention provides methods for treating or preventing a Condition in a subject comprising administering to the subject an effective amount of fucosylated chondroitin sulfate and an effective amount of a COX-2 inhibitor.

In a further embodiment, the present invention provides methods for treating or preventing a Condition in a subject comprising administering to the subject an effective amount of fucosylated chondroitin sulfate and an effective amount of a matrix metalloproteinase inhibitor.

In yet another embodiment, the present invention provides methods for treating or preventing a Condition in a subject comprising administering to the subject an effective amount of fucosylated chondroitin sulfate and an effective amount of resveratrol.

In yet another embodiment, the present invention provides methods for treating or preventing a Condition in a subject comprising administering to the subject an effective amount of fucosylated chondroitin sulfate and an effective amount of 12-methyltetradecanoic acid.

Effective amounts of the other therapeutic agents are well known to those skilled in the art. However, it is well within the skilled artisan's purview to determine the other therapeutic agent's optimal effective amount range. In one embodiment of the invention, where, another therapeutic agent is administered to a subject, the effective amount of the Compound or Extract of the Invention is less than its effective amount would be where the other therapeutic agent is not administered. In this case, without being bound by theory, it is believed that the Compounds and Extracts of the Invention and the other therapeutic agent act synergistically to treat or prevent a Condition.

### 5.5 Kits

The invention encompasses kits that can simplify the administration of a Compound or Extract of the Invention to a subject.

A typical kit of the invention comprises a unit dosage form of a Compound or Extract of the Invention. In one embodiment the unit dosage form is a container, which can be sterile, containing an effective amount of a Compound or Extract of the Invention, and a physiologically acceptable carrier or vehicle. The kit can further comprise a label or printed instructions instructing the use of the Compound or Extract of the Invention to treat or prevent a Condition in a subject. The kit can also further comprise a unit dosage form of another therapeutic agent, for example, a container containing an effective amount of the other therapeutic agent. In one embodiment the kit comprises a container containing an effective amount of a Compound or Extract of the Invention and an effective amount of another therapeutic agent. Examples of other therapeutic agents include, but are not limited to, those listed above.

Kits of the invention can further comprise a device that is useful for administering the unit dosage forms. Examples of such a device include, but are not limited to, a syringe, a drip bag, a patch, an inhaler, and an enema bag.

The following examples are set forth to assist in understanding the invention and should not, of course, be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### 6. EXAMPLES

### 6.1 Example 1

### Isolation of Fucosylated Chondroitin Sulfate from Sea Cucumber Body Wall

Fucosylated chondroitin sulfate can be isolated from the body wall of the sea cucumber and purified according to the method described in Mourao et al., J Biol Chem., 271:23973-84 (1996).

### 6.2 Example 2

### Extraction of Catechins from Green Leaves

Crude catechins can be extracted from Chinese green tea leaves (500 g) using hot water, as described in European Patent No. EP 1402869 to Schneider, then dissolved in ethanol (5 mL). The ethanolic solution is then loaded onto a C16/100 chromatographic column (1.6 cm×90 cm, Sephadex LH-20, equilibrated using ethanol) and chromatographed using a flow rate of about 1.2 mL/min. Collected fractions can be first analyzed using thin-layer chromatography on silica gel plates (chloroform/methanol/water (65:35:10, v/v/v as eluent) and subsequently developing the eluted plates using a spray reagent that is prepared by dissolving 1 g vanillin in 50 mL concentrated HCl. Fractions that are positive to the spray reagent can be concentrated in vacuo, and the resulting residue dissolved in methanol. The resulting methanolic solution can be analyzed using UV/visible spectroscopy by measuring their absorbances at 280 nm and 500 nm for detecting the presence of catechins. Fractions containing catechins can be purified using the methods described in Hoefler et al., J. Chromatogr. 129:460-3 (1976).

### 6.3 Example 3

### Preparation of 12-Methyltetradecanoic Acid

12-Methyltetradecanoic Acid can be obtained from a sea cucumber extract (Cucumaria frondosa) and purified according to the method set forth in U.S. Pat. No. 6,055,936 to Collin.

### 6.4 Example 4

### Preparation of (−)-Epigallocatechin and (−)-Epigallocatechin Gallate

(−)-Epigallocatechin can be commercially obtained, for example, from Zhejiang Yixin Pharmaceutical Co., Ltd. (Zhejiang, China).

(−)-Epigallocatechin gallate can be commercially obtained, for example, from Sigma-Aldrich (St. Louis, Mo.).

### 6.5 Example 5

### Preparation of Extract 1

The whole body wall of a sea cucumber was isolated as described in U.S. Pat. No. 5,985,330 to Collin. The body wall thus obtained was then soaked in a solution of 10% Alcalase (NOVO Nordisk Bio Chem, North Carolina) in fresh water at a temperature of 130° F. (±30° F.) for about one hour, then processed by hand to further isolate the black epithelial layer of the body wall from the underlying collagenous tissues. The black epithelial layer thus obtained was dried using a 40 hp “heat pump” dryer (Southwind Mfg., Nova Scotia, Canada) to a moisture content of about 3% moisture, then finely divided to obtain a powder. The powder obtained was finely divided, then diluted with water and the resultant solution was allowed to stir for about 12 hours at room temperature, then centrifuged at 30,000 RPM for about 1 hour. The resultant supernatant was removed and lyophilized to provide the Extract 1 as a powder.

### 6.6 Example 6

### Preparation of Extract 2

Extract 1, prepared using the procedure set forth in Example 5 above, was diluted with 70% aqueous isopropanol (3:1 dilution by volume) and the resultant solution was stirred at room temperature for about 30 hours. The solution was then filtered, and the filtrate was concentrated in vacuo to provide Extract 2 as a powder.

### 6.7 Example 7

### Preparation of Extract 3

During the processing operation of removing viscera and muscle set forth in Example 5, the anterior portion of the sea cucumber Cucumaria frondosa was removed, including the mouth portion of the head with surrounding tentacles. This anterior portion was then diluted with water and the resultant solution was heated to reflux and allowed to remain at this temperature for about 30 minutes. The resultant mixture was allowed to cool to room temperature and was then filtered and the collected sea cucumber body portion was dried in a conventional heat-pump dryer as described in Example 5 to provide a powder. The powder obtained was finely divided, then diluted with water and the resultant solution was allowed to stir for 12 hours at room temperature, then centrifuged at 30,000 RPM for one hour. The resultant supernatant was removed and lyophilized to provide the Extract 3 as a powder.

### 6.8 Example 8

### Preparation of Extract 4

The powder obtained in Example 6 was diluted with hexane and the resultant solution was heated to reflux for about 2 hours, then cooled to room temperature, filtered and concentrated in vacuo to provide Extract 4 as a powder.

### 6.9 Example 9

### Preparation of Extract 5

Sea cucumber intestinal tissue was subjected to supercritical carbon dioxide extraction at a pressure of 4000 psi, an extraction temperature of 60° C. and a flow rate of 1.5 ml per minute to provide Extract 5 as a powder. The resultant extract had no MMP-2 inhibitory ability.

### 6.10 Example 10

### Preparation of Extract 6

The whole body wall of a sea cucumber was isolated as described in U.S. Pat. No. 5,985,330 to Collin. The body wall thus obtained was then soaked in boiling water for about one hour, then dried using a 40 hp “heat pump” dryer (Southwind Mfg., Nova Scotia, Canada) to a moisture content of about 3% moisture. The dried body wall was then finely divided to obtain a first powder, which was diluted with water. The resultant solution was allowed to stir for about 12 hours at room temperature, then centrifuged at 30,000 RPM for about 1 hour. The resultant supernatant was removed and lyophilized to provide a second powder. The second powder was than diluted with ethanol and the resultant solution was heated to reflux and allowed to stir for about 10 hours at reflux, then cooled to room temperature. The cooled solution was filtered and the filtrate was concentrated in vacuo to provide Extract 6 as a powder.

### 6.11 Example 11

### Preparation of Composition 1

Composition 1 was prepared by combining 40% eicosapentaenoic acid, 10% of a commercial green tea extract (containing 95% polyphenols), 10% sea cucumber ethanol extract of epithelial layer, 5% boswellia, and 5% resveratrol from Japanese knotweed (polygonum cuspidatum). The remainder of the composition consisted of fish oil triglycerides.

### 6.12 Example 12

### Determination of the Effect of Illustrative Compounds and Extracts of the Invention on the Activity of MMP-2 in Fibroblasts Cultured from AAA Tissue Specimens

Properties of different test compounds against MMP-2 activity of aneurysmal fibroblast origin were examined on 10% polyacrylamide gels containing 1.0 mg/mL of gelatin. Fibroblasts cultured from AAA specimens were lysed using the CellLytic-M lysis/extraction reagent. The protein concentration of each supernatant was determined using the Bradford method using bovine serum albumin as a standard. The aortic fibroblast lysates were incubated alone (control) or with an illustrative Compound or Extract of the Invention at various concentrations for 1 hour on ice. After test compound incubation, equivalent amounts of proteins (30 μg) were mixed with an equal volume of sample buffer (2% SDS, 125 mM Tris-HCL, pH 6.8, 10% glycerol and 0.001% bromophenol blue) and the resulting mixture was subjected to electrophoresis in Trir-glycine el. buffer (25 mMTris pH 8.3, 250 mM Glycine, 0.1% SDS) under denatured but non-reduced conditions. After completion of electrophoresis, the gels were incubated in 2% Triton X-100 for 30 min at room temperature to ensure complete removal of SDS. The gels were then incubated at 37° C. overnight in development buffer (0.005M Tris-HCl (pH 7.5), 0.2MNaCl, 0.005MCaCl₂, and 0.02% Brij-35) (Bio-Rad). Gels were stained with 0.05% Coomassie blue R250 (Bio-Rad) for 30 min and destained twice in a mixture of 40% methanol and 10% acetic acid for 20 min. Enzyme activity was detected as unstained bands on a blue background. Quantity One (Bio-Rad Version 4.3.1) was used as a densitometer to quantitate test compound inhibitory ability.

Table 3 shows the relative MMP-2 inhibitory activity of illustrative Extracts and Compounds of the Invention.

TABLE 3
Relative MMP-2 inhibitory activity of illustrative Extracts and
Compounds of the Invention

| Sample | MMP-2 Inhibitory Activity |
|---|---|
| fucosylated chondroitin sulfate | +++ |
| (−)-epigallocatechin gallate | +++ |
| (−)-epigallocatechin | + |
| 12-methyltetradecanoic acid | + |
| (−)-epigallocatechin gallate and | +++ |
| fucosylated chondroitin sulfate | |
| (1:1 mixture)* | |
| Extract 1 | − |
| Extract 2 | +++ |
| Extract 4 | − |
| Extract 5 | − |
| Composition 1 | +++ |

+++ indicates a very high degree of MMP-2 inhibition relative to the other compounds tested
++ indicates a moderate degree of MMP-2 inhibition relative to the other compounds tested
+ indicates a minor degree of MMP-2 inhibition relative to the other compounds tested
− indicates that the tested compound did not inhibit MMP-2
*1:1 mixture by weight

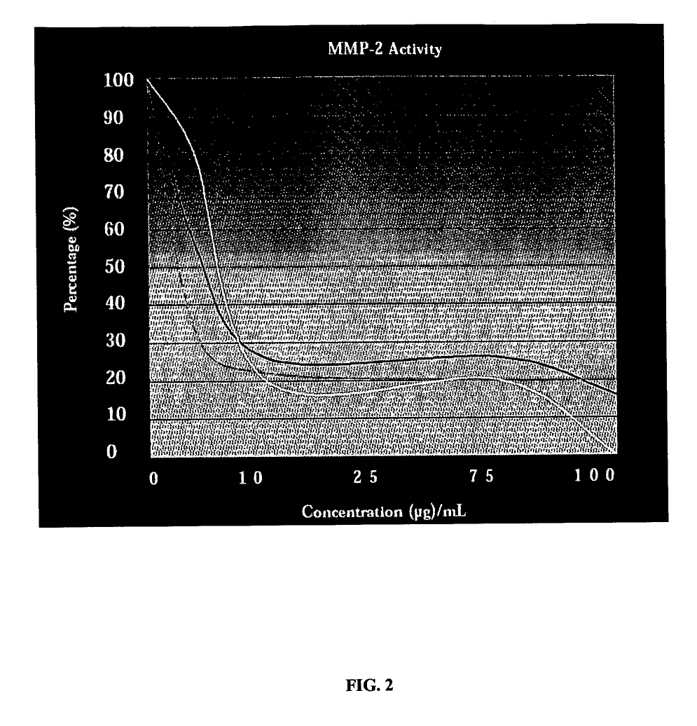

Results obtained in connection with (−)-epigallocatechin gallate, fucosylated chondroitin sulfate, and a 1:1 mixture (by weight) of (−)-epigallocatechin gallate and fucosylated chondroitin sulfate are also in graphical form in FIG. 2.

These results indicate that fucosylated chondroitin sulfate, (−)-epigallocatechin gallate, (−)-epigallocatechin, 12-methyltetradecanoic acid, Extract 2, and Composition 1, illustrative Compounds and Extracts of the Invention, inhibit aneurysmal fibroblast MMP-2 activity. Accordingly, this demonstrates that the Compounds and Extracts of the Invention are useful for treating an aneurysm.

### 6.13 Example 13

### Inhibition of Total Complement by Fucosylated Chondroitin Sulfate

Blood was collected from a healthy human donor and allowed to clot at room temperature for 60 minutes. The serum was separated from the clotted blood and transferred to a clean tube. Fucosylated chondroitin sulfate (FCS) was incubated in concentrations of 0.1 mg/mL, 0.4 mg/mL and 1 mg/mL for 30 minutes at 37° C. and the complement was examined by assaying total complement activity (CH₅₀). Complement inhibition was about 40% at a FCS concentration of 0.1 mg/mL, about 83% at a concentration of 0.4 mg/mL and complete at a concentration of 1.0 mg/mL.

Since complement activation is one etiologic pathway of AAA pathology, this experiment indicates that FCS, an illustrative Compound of the Invention, is useful for the treatment of AAA.

### 6.14 Example 14

### Inhibition of Total Complement by Fucosylated Chondroitin Sulfate

An experiment to measure the complement factors C3a and C5a was carried out at 63° C. for 30 minutes using heat-aggregated gamma globulin (HAGG, at 14 mg/mL), a complement activator with known activity, as a positive control.

Four separate mixtures were prepared by mixing 8 parts of normal human serum with: (1) 2 parts saline, (2) 1 part saline and 1 part HAGG, (3) 1 part saline and 1 part fucosylated chondroitin sulfate (FCS), or (4) 1 part HAGG and 1 part FCS. These four mixtures were then incubated for 30 minutes at 37° C. and the complement was examined by assaying for levels of C3a and C5a. Results indicate that FCS has a strong inhibitory activity toward both C3a and C5a, both alone and with the classic complement pathway inhibitor HAGG.

Since complement activation is one etiologic pathway of AAA pathology, this experiment indicates that FCS, an illustrative Compound of the Invention, is useful for the treatment of AAA.

### 6.15 Example 15

### Determination of In Vivo Efficacy of the Compounds and Extracts of the Invention against Abdominal Aortic Aneurysm

After being allowed to become accustomed to their caged environment for seven days, twenty-four laboratory animals (chosen from Sprague-Dawley rats or C57BLA mice), denoted as Set A, are anesthetized (ketamine 50-70 mg/kg and xylazine 5 mg/kg IP) and subjected to an abdominal middle line laparotomy, resulting in exposure of the abdominal aorta. Selected animals in Set A (those in groups II, III, and IV, described below) will then have cotton gauze strips that have been previously soaked in solution of 5 mL calcium chloride (13.6 mEq calcium/10 mL) and 5 mL sodium chloride (0.9% NaCl) for 20 minutes, placed directly on the abdominal aorta at approximately the center of the section between the renal artery and the iliac bifurcation.

A second group of 24 animals, denoted as Set B, is then anesthetized (ketamine 50-70 mg/kg and xylazine 5 mg/kg IP) and each animal is rotated to the right lateral decubitus position, thereby exposing the animal's left chest. A lateral incision is made in the thorax and through this incision, via the fifth intercostal space, the descending thoracic aorta is exposed. Selected animals in Set B (those in groups II, III, and IV, described below) will then have cotton gauze strips that have been previously soaked in solution of 5 mL calcium chloride (13.6 mEq calcium/10 mL) and 5 mL sodium chloride (0.9% NaCl) for 20 minutes, placed directly on the thoracic aorta. The incisions are closed, the animals are allowed to recover, and are then placed in cages for follow-up. The animals are then administered the analgesic agent buprenorphine (0.5-1.0 mg/kg SQ every 8-12 hours during the first postoperative day). Four weeks later the animals are humanly sacrificed (100% CO₂ to effect and then pentobarbital 100 mg/kg IP) and the dilated aortic segments are harvested for histologic, biochemical, immunohistochemical, and molecular biological analyses.

In carrying out the experiment, the animals in Set A will be divided into 4 groups: group I (n=6) sham operation, in which no treated gauze strips are placed on the abdominal aorta; group II (n=6) in which calcium chloride soaked gauze strips will be placed on the abdominal aorta as described above; group III (n=6) in which calcium chloride soaked gauze strips will be placed on the abdominal aorta as described above, and in addition, each animal will receive a postoperative administration of a Compound or Extract of the Invention; and group IV (n=6) in which calcium chloride soaked gauze strips will be placed on the abdominal aorta as described above, and in addition, each animal will receive a postoperative administration of doxycycline, a known MMP-2 inhibitor.

The animals in Set B will be also be divided into the same 4 groups (I-IV) as described in the previous paragraph for Set A.

### 6.16 Example 16

### Determination of In Vivo Efficacy of an Extracts of the Invention Against Abdominal Aortic Aneurysm

C57BLA mice were administered a green tea extract containing 55% (−)-epigallocatechin gallate via drinking water (dosage of 250 mg/kg/d) beginning one week preoperatively. Abdominal aortic dilatation was induced described in Example 15, in both control (n=17) and treated mice as (n=16) using 0.25M and 0.5M CaCl₂. Sham mice (n=5) underwent the procedure with normal saline only. Digital video analysis was used to calculate in vivo aortic diameter at initial surgery and at the time of sacrifice. At 4 weeks post-induction, the abdominal aorta was perfusion fixed and sectioned for histologic analysis. Statistical analysis was performed with Paired Student's t-Test and One-Way ANOVA. Post hoc analysis was completed with the Tukey Test.

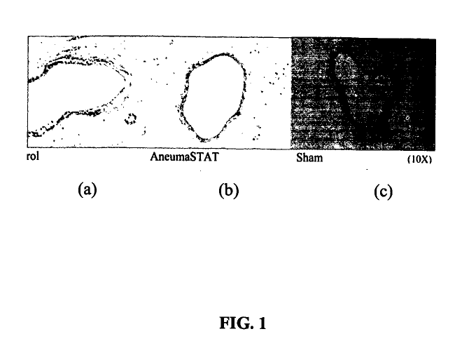

The results indicate that no difference in aortic size was noted between mice induced with 0.25M and 0.5M CaCl₂ Control mice demonstrated a significant growth in aortic diameter at sacrifice compared to the initial size (532 vs. 768 μm, p<0.001). In animals treated with the green tea extract, no significant increase in aortic size was noted (550 vs. 580 μm, p>0.05). Similarly, Sham mice demonstrated no significant growth in aortic diameter (478 vs. 477 μm, p>0.05). There was no significant difference in the initial aortic diameter among the three groups (p>0.05). At sacrifice, and as illustrated in FIG. 1a-c, the aortic diameters of the green tea extract treated group and Sham group were not significantly different (p>0.05). However, significant differences were noted when compared individually to the Control group (p=0.005 and p=0.003, respectively). Histologic examination revealed disorganization of the elastin and collagen fibrils, fragility of the media, and inflammation of the adventitia in Control mice, whereas the green tea extract treated group and Sham groups appeared relatively normal.

These results demonstrate that an illustrative green tea extract effectively prevented aneurysm growth in an animal model and accordingly, the extracts of the invention are useful for treating or preventing an aneurysm.

The present invention is not to be limited in scope by the specific embodiments disclosed in the examples, which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

What is claimed is:

1. A method for treating or preventing an aneurysm in a subject, the method comprising administering to the subject an effective amount of a compound or pharmaceutically acceptable salt of a compound selected from (+)-catechin, (−)-epicatechin, (−)-epicatechin gallate, (−)-gallocatechin gallate, (−)-epigallocatechin or epigallocatechin gallate, wherein the compound is in isolated form.

2. A method for treating or preventing a cardiovascular disease in a subject, the method comprising administering to the subject an effective amount of a compound or pharmaceutically acceptable salt of a compound selected from (+)-catechin, (−)-epicatechin, (−)-epicatechin gallate, (−)-gallocatechin gallate, (−)-epigallocatechin or epigallocatechin gallate, wherein the compound is in isolated form.

3. A method for treating or preventing a peripheral vascular disorder in a subject, the method comprising administering to the subject an effective amount of a compound or pharmaceutically acceptable salt of a compound selected from (+)-catechin, (−)-epicatechin, (−)-epicatechin gallate, (−)-gallocatechin gallate, (−)-epigallocatechin or epigallocatechin gallate, wherein the compound is in isolated form.

4. The method of any one of claims 1-3, wherein the compound is (−)-epigallocatechin gallate.

5. A method for treating or preventing an aneurysm in a subject, the method comprising administering to the subject an effective amount of a sulfated polysaccharide or a pharmaceutically acceptable salt thereof, wherein the sulfated polysaccharide is in isolated form.

6. A method for treating or preventing a cardiovascular disease in a subject, the method comprising administering to the subject an effective amount of a sulfated polysaccharide or a pharmaceutically acceptable salt thereof, wherein the sulfated polysaccharide is in isolated form.

7. A method for treating or preventing a peripheral vascular disorder in a subject, the method comprising administering to the subject an effective amount of a sulfated polysaccharide or a pharmaceutically acceptable salt thereof, wherein the sulfated polysaccharide is in isolated form.

8. The method of any one of claims 5-7, wherein the sulfated polysaccharide is fucosylated chondroitin sulfate.

9. The method of claim 1 or 5, wherein the aneurysm is an abdominal aortic aneurysm.

10. The method of any one of claims 1-3, further comprising administering to the subject an effective amount of fucosylated chondroitin sulfate.

11. The method of any one of claims 5-7, further comprising administering to the subject an effective amount of (−)-epigallocatechin gallate.

12. A method for treating or preventing an aneurysm in a subject, the method comprising administering to the subject an effective amount of a green tea extract and an effective amount of a sea cucumber extract.

13. A method for treating or preventing a cardiovascular disease in a subject, the method comprising administering to the subject an effective amount of a green tea extract and an effective amount of a sea cucumber extract.

14. A method for treating or preventing a peripheral vascular disorder in a subject, the method comprising administering to the subject an effective amount of a green tea extract and an effective amount of a sea cucumber extract.

15. The method of claim 12, further comprising the administration of one or more of 12-methyltetradecanoic acid, resveratrol, tensilin, boswellia, eicosapentaenoic acid, and glucosamine hydrochloride.

16. The method of claim 12, further comprising the administration of boswellia, resveratrol, eicosapentaenoic acid, and fish oil triglycerides.

17. The method of claim 12, further comprising the administration of glucosamine hydrochloride.

18. The method of claim 12, wherein the green tea extract contains about 95% polyphenols and the sea cucumber extract is prepared using a process comprising:
(i) isolating the whole body wall of a sea cucumber;
(ii) soaking the isolated whole body wall in an aqueous solution;
(iii) processing the whole body wall to further isolate the epithelial layer of the body wall from the underlying collagenous tissues;
(iv) drying the epithelial layer to a water content of less than 3%;
(v) grinding the dried epithelial layer to provide a powder;
(vi) diluting the powder with water and centrifuging the resultant solution; and
(vii) drying the resultant supernatant to provide the sea cucumber extract as a powder.

19. The method of claim 12, wherein the green tea extract contains about 95% polyphenols and the sea cucumber extract is prepared using a process comprising:
(i) isolating the anterior portion of a sea cucumber;
(ii) soaking the isolated anterior body portion in an aqueous solution;
(iii) drying the isolated anterior body portion to a water content of less than 3%;
(iv) grinding the dried anterior body portion to provide a powder;
(v) diluting the powder with water and centrifuging the resultant solution; and
(vi) drying the resultant supernatant to provide the sea cucumber extract as a powder.

20. The method of claim 12, wherein the green tea extract contains about 95% polyphenols and the sea cucumber extract is prepared using a process comprising:
(i) isolating the whole body wall of a sea cucumber;
(ii) soaking the isolated whole body wall in an aqueous solution;
(iii) drying the whole body wall to a water content of less than 3%;
(iv) grinding the dried body wall to provide a powder;
(v) diluting the powder with water and centrifuging the resultant solution; and
(vi) drying the resultant supernatant to provide the sea cucumber extract as a powder.
